(19) United States
(12) Patent Application Publication
(10) Pub. No.: US 2022/0323629 A1
(43) Pub. Date: Oct. 13, 2022

(54) Method And Device For Destructive Removal Of Odors From An Animal Litter Box

(71) Applicant: Xenex Disinfection Services Inc., San Antonio, TX (US)

(72) Inventors: Gregory Fridman, Philadelphia, PA (US); Charles Christopher Bailey, III, Philadelphia, PA (US)

(21) Appl. No.: 17/838,379

(22) Filed: Jun. 13, 2022

Related U.S. Application Data
(63) Continuation of application No. PCT/US2020/064733, filed on Dec. 13, 2020.
(60) Provisional application No. 62/947,586, filed on Dec. 13, 2019.

(51) Int. Cl. A61L 9/015; A01K 1/01; A01K 1/00

(52) U.S. Cl. CPC A61L 9/015; A01K 1/0107; A01K 1/0047

(57) ABSTRACT

Animal litter boxes are provided which include a sensor to detect a change in an environment within an interior of its housing and an odor removal device (ORD) operationally connected to the sensor such that the ORD is activated in response to a signal from the sensor. In some cases, the ORD is configured to operate in different manners based on respectively different signals from the sensor. In addition or alternatively, the litter boxes may include a timer configured with the sensor for activating and periodically reactivating the ORD at preset time intervals in response to a sensor signal. In yet other embodiments, the litter boxes may additionally or alternatively include a timer having user adjustable settings of different time periods at which to initiate activation of the ORD and/or user adjustable settings of different durations at which to operate the ORD, both in response from a sensor signal.

### PRIORITY CLAIM

The present application is a continuation application of International Application No. PCT/US2020/064733 filed Dec. 13, 2020, which designates the United States and claims priority to U.S. Provisional Patent Application No. 62/947,586 filed Dec. 13, 2019.

### BACKGROUND OF THE INVENTION

### 1. Field of the Invention

The present invention relates to an animal litter box, devices for removing odors, and more particularly relates to a method and device for eliminating odors produced by an animal while using the litter box.

### 2. Description of the Related Art

Domesticated animals of a vast range of species are trained, out of convenience or necessity, to eliminate their urine and feces, hereinafter referred to collectively as “excreta,” within litter box containers, usually lined with an absorbent disposable material such as a clay-based or similar litter filler material. Litter boxes are frequently stored indoors in an animal owner's home. Consequently, the unpleasant odors naturally produced by the excreta permeate outside the litter box into the animal owner's household. The odors are created by volatile organic compounds (VOCs) and other odor-producing chemicals released when an animal eliminates its excreta. These odor producing chemicals can linger within the litter box and throughout the household for significant periods of time, even after the owner has disposed of the litter box lining. Moreover, the disposal of the litter box lining typically occurs after the animal has used the litter box numerous times, allowing for the buildup of odors within and outside of the litter box.

Litter box ventilation systems designed to remove and eliminate these unwanted odors produced by animal excreta are not new. These ventilation systems consist of some configuration of an exhaust fan connected to the housing to pull air from within the litter box and expel it outside the litter box, and often, but not necessarily, contain an air filter. These ventilation systems take air from within the litter box and dispel it outside the litter box, transferring the odors into the animal owner's home without eliminating the source of these odors. The addition of a filter to a ventilation system can trap odor producing compounds and relieve the animal owner temporarily from the unpleasant odors, but the filter quickly becomes too full to absorb any more compounds. The animal owner must change the filter frequently to remove the odor from the household, but odors can linger in the litter box and in the area outside the litter box even after the disposal of the filter. The present invention solves this problem through the addition of a destructive odor removal device that eliminates the chemical source of the unpleasant odors.

### SUMMARY OF THE INVENTION

The present disclosure relates to a device and method for eliminating unwanted odors from an animal litter box. The following description of various embodiments of the apparatuses and methods is not to be construed in any way as limiting the subject matter of the appended claims.

Animal litter boxes are provided which include a sensor to detect a change in an environment within an interior of its housing and an odor removal device operationally connected to the sensor such that at least one component of the odor removal device is activated in response to a signal from the sensor. In some cases, the odor removal device is configured to operate in different manners based on respectively different signals from the sensor. In addition or alternatively, the litter boxes may include a timer configured with the sensor for activating and periodically reactivating the odor removal device at preset time intervals in response to a sensor signal. In yet other embodiments, the litter boxes may additionally or alternatively include a timer having user adjustable settings of different time periods at which to initiate activation of the odor removal device and/or user adjustable settings of different durations at which to operate the odor removal device, either of which is in response from a sensor signal.

In an embodiment, an animal litter box includes a housing having an opening, a sensor to detect a change in an environment, an odor removal device comprising a motorized fan for passing air into the odor removal device, a pre-filter, a plasma generating unit, an activated carbon filter, an air inlet vent, and an exhaust vent. The sensor is operationally connected to the odor removal device, wherein at least one component of the odor removal device is activated in response to a signal from the sensor.

In a further embodiment, the sensor is operationally connected to the fan and/or the plasma generating device.

In a further embodiment, the animal litter box further includes a timer with preset time delay periods to delay the activation of the at least one component of the odor removal device.

In a further embodiment, the at least one component of the odor removal device is configured to operate in different manners based on respectively different signals from the sensor.

In a further embodiment, the at least one component of the odor removal device is the motorized fan and the motorized fan is configured to operate at different speeds and/or for different durations based on respectively different signals from the sensor.

In a further embodiment, the at least one component of the odor removal device is the plasma generating unit and the plasma generating unit configured to operate with different power levels and/or for different durations based on respectively different signals from the sensor.

In a further embodiment, an animal litter box includes a housing having an opening allowing for entry and exit of an animal and an odor removal device at least partially disposed on the housing. The odor removal device includes an air inlet vent fluidly connected to an interior of the housing, a motorized fan for passing air from an interior of the housing into the air inlet vent, a plasma generating unit, an air filter disposed between the air inlet vent and the plasma generating unit, an exhaust vent, and an activated carbon filter disposed between the plasma generating unit and the exhaust vent.

In a further embodiment, the animal litter box further includes a sensor to detect a change in an environment.

In a further embodiment, an animal litter box comprises a housing having an opening allowing for entry and exit of an animal, a sensor for determining when an animal has exited the housing, and an odor removal device. The sensor is operationally connected to the odor removal device so that at least one component of the odor removal device is activated after receipt of a signal from the sensor indicating an animal has exited the housing.

In a further embodiment, the at least one component of the odor removal device is a motorized fan.

In a further embodiment, the at least one component of the odor removal device is a plasma generating unit.

In a further embodiment, the at least one component of the odor removal device is an ozone generator.

In a further embodiment, the odor removal device further includes an activated carbon filter.

In a further embodiment, the sensor is selected from the group consisting of a motion sensor, a moisture sensor, an image sensor, a heat sensor, a chemical sensor, a pH sensor, a light sensor, a weight sensor, a pressure sensor, and combinations thereof.

In a further embodiment, the activated carbon filter is a silver-impregnated activated carbon filter.

In a further embodiment, the animal litter box includes at least one wall and the opening is on the at least one wall.

In a further embodiment, the housing includes at least four walls and the opening is in one of the walls of the housing allowing for entry and exit of an animal.

In a further embodiment, the animal litter box includes a top wall, a front wall, a back wall, and two side walls, wherein the top wall is connected to a top interior edge of the back wall by a hinge mechanism and the remaining three side walls of the top wall are disconnected from a top edge of the other three adjacent the walls to allow for movement of the top wall away from the housing.

In a further embodiment, the animal litter box includes a top wall, a front wall, a back wall, and two side walls, wherein a back edge of the back wall is connected to a top edge of the bottom wall by a hinge mechanism and the back wall is disconnected from the other three adjacent walls to allow for movement of the back wall away from the housing.

In a further embodiment, the odor removal device is removable from the housing.

In a further embodiment, the housing includes a bottom wall, wherein a top face of the bottom wall has at least one projection of height equal to or less than the height of a container to be received within the housing.

In a further embodiment, the container comprises raised edges.

In a further embodiment, a method for removing volatile organic compounds within an animal litter box includes the steps of detecting, with a sensor, a change in an environment and activating a motorized fan to initiate a flow of air from within the animal litter box into an odor removal device comprising a plasma generating unit. The method further includes activating the plasma generating unit to remove an odor producing chemical contained in the flow of air, routing air released from the plasma generating unit through an activated carbon filter, and releasing air from the activated carbon filter.

In a further embodiment, activating the motorized fan is conducted upon the sensor detecting a change in the environment or after a preset delay after the sensor detects a change in the environment.

In a further embodiment, the activated carbon traps trace organic compounds and remaining plasma from the flow of air.

In a further embodiment, the step of releasing air from the activated carbon filter includes releasing the air into the animal litter box.

In a further embodiment, the step of releasing air from the activated carbon filter includes releasing the air to an area outside the animal litter box.

In a further embodiment, the change in an environment is a movement of an animal within the housing.

In a further embodiment, activating the motorized fan is conducted after no movement and/or occupancy is detected within the animal litter box for a predetermined amount of time.

In a further embodiment, the animal litter box includes a waste receiving material disposed of within the animal litter box for receiving excreta.

In a further embodiment, the method further comprises terminating operation of the motorized fan and the plasma generating unit, and thereafter, periodically reactivating the motorized fan and the plasma generating unit for predetermined time intervals until the sensor detects a change in the environment.

In a further embodiment, an odor removal device comprises an air inlet vent, a motorized fan for passing air into the air inlet vent, a plasma generating unit, and an air filter disposed between the air inlet vent and the plasma generating unit. The odor removal device further includes an exhaust vent and an ion exchange element, for example, an activated carbon filter disposed between the plasma generating unit and the exhaust vent.

In a further embodiment, the odor removal device further includes a sensor operationally connected to the motorized fan and/or the plasma generating unit to activate the motorized fan and/or the plasma generating unit in response to a signal from the sensor.

In a further embodiment, the litter box contains a housing, an opening for an animal to enter and exit the housing, a container for an animal to eliminate its excreta into, and an odor removal device for detecting the presence of an animal in the housing and for the destructive elimination of the odor producing chemicals produced by animal excreta. The litter box further includes two vents within the odor removal device to allow for the entrance of air into the odor removal device and exit of the cleaned and filtered air out of the odor removal device.

In a further embodiment, the odor removal device is affixed to or implanted in one of the walls of the housing and includes a sensor to detect movement within the housing, namely, for detecting the presence of an animal, a fan, a plasma generating unit, a pre-filter, a silver-impregnated activated carbon filter, and two vents. The two vents include an air inlet vent to allow travel of air into the odor removal device and an exhaust vent to allow travel of air out of the odor removal device. In the various embodiments, a sensor detects a change in an environment, such as the entry or exit of an animal within the housing of the litter box. The activation of the sensor triggers the motorized fan and the plasma generating unit to turn on. The sensor could be, but is not limited to, a motion sensor, heat sensor, entry or exit sensor, occupancy sensor, photo sensor, an odor sensor, a pH sensor, or volatile compound sensor, an image sensor or other sensor capable of detecting an animal and activating the odor removal device. The fan could be, but is not limited to, a mechanical fan of any size powered by AC current, battery or solar.

In an embodiment, the fan and the plasma generating unit within the odor removal device are configured to be triggered to turn on by the sensor after a fixed time interval, determined by a timer configured with the sensor, within moments of the animal exiting the litter box. The delay between the exit of the animal exiting the litter box and the activation of the fan and the plasma generating unit is necessary to prevent the startling or distraction of the animal inside the litter box by the noise of the odor removal device. If desired, the timer configured with the sensor can be configured to delay the triggering of the fan and the plasma generating unit for set time intervals after the use of the litter box by the animal to eliminate additional odors produced over time by the excreta present in the litter box.

In an embodiment, upon receiving a signal from the sensor, a fan within the odor removal device is powered on and draws air from within the housing of the litter box into the odor removal device through the top air inlet vent. A pre-filter is located on the interior side of the top air inlet vent to trap large debris in the air such as household dirt, animal dander, hair, fur, or feathers to prevent their entrance into the odor removal device and potential damage to the component parts. The plasma generating unit is also activated by the same signal from the sensor and, when activated, produces plasma containing positive ions, negative ions, and reactive species that break down and eliminate the odor producing chemicals produced by animal excreta, including VOC's among other chemicals, that travel in the air drawn into the odor removal device. The electronic components of the odor removal device, including the sensor, the fan, and the plasma generating unit are powered either by a direct current (DC) power supply, an alternating current (AC) power supply, or by a battery, either disposable alkaline or rechargeable batteries.

In a further embodiment, after the reactive species produced by the plasma generating unit have eliminated the chemical compounds that produce odors from the animal excreta, some chemical byproducts are produced, including ozone, that can cause adverse health effects in humans and are undesirable to members of the household in which the invention resides. To prevent the dispersal of the byproducts produced by the plasma generating unit into the general area outside the box, an adsorptive material for capturing impurities such as an ion exchange element, such as a resin or beads, or an activated carbon, such as a removable silver-impregnated activated carbon filter is built into the odor removal device below the plasma generating unit. As the air that has been pulled into the odor removal device travels through the odor removal device, the odor-producing chemical compounds are eliminated and byproducts are trapped in the silver-impregnated activated carbon filter. The carbon filter will eventually run out of active sites for absorption of chemical byproducts and will need to be replaced periodically. The owner of the litter box will be instructed to periodically replace this carbon filter, based on their animals' use of the litter box.

The litter box may further comprise a disposable container containing a waste receiving material, such as an absorbent, granular material. The container is used for collection of animals' excreta and the absorbent, granular material is used for the absorption of excreta to make it more desirable for the animal to use the litter box multiple times between cleanings of the litter box and easier for the owner to clean the litter box.

Other features and advantages of the invention will appear from the following description in which the preferred embodiments have been set forth in detail, in conjunction with the accompanying drawings.

### BRIEF DESCRIPTION OF THE FIGURES

FIG. 1 is a cross-sectional perspective of an embodiment of the litter box device constructed in accordance with the principles of the present invention.

FIG. 2 is an exploded view of an embodiment of the housing and removable or disposable container or tray construction of the present invention.

FIG. 3 is a side cutaway view of an embodiment of the present invention depicting the odor removal device mounted on a wall of the housing with a vent built into the housing wall adjacent the odor removal device.

FIG. 4 is a side cutaway view of an embodiment of the present invention depicting the odor removal device mounted directly in an opening of the of the housing.

FIG. 5 is a schematic block diagram of a method for removing odors from a litter box.

FIG. 6 is perspective view of an embodiment of the present invention in which the top wall is connected to the back wall by a hinge and can open away from the housing.

FIG. 7 is perspective view of an embodiment of the present invention in which the back wall is connected to the top wall by a hinge and can open away from the housing.

FIG. 8 is an exemplary diagram depicting the flow of air into the odor removal device and passing through the components of the odor removal device, including the sensor, fan, pre-filter, plasma generating unit, and the silver-impregnated activated carbon filter, and out the exhaust vent.

The same reference numerals refer to the same parts through the various figures.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

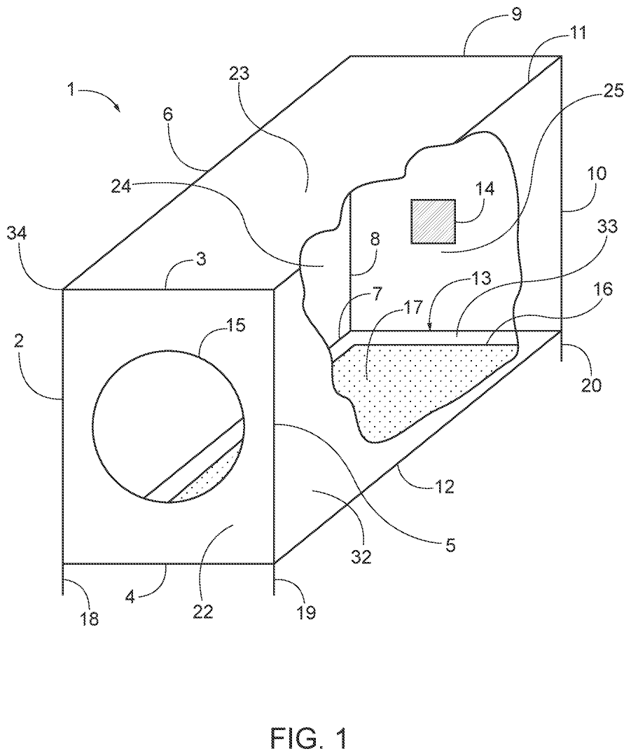

With reference now to the drawings, in particular FIGS. 1 through 8 thereof, an embodiment of the new and improved litter box 1 embodying the principles and concepts of the present invention will be described.

Specifically, it will be noted in the various figures that the device relates to a litter box 1 for eliminating animal excreta odors within an enclosed space. In its broadest context, the litter box 1 consists of a housing for receiving an animal. The litter box further comprises an odor removal device containing a sensor, and two openings within the odor removal device: one to allow for the entrance of air from within the housing into the device and one for the escape of filtered air. Such components are individually configured and correlated with respect to each other so as to attain the desired objective. Preferably, the device is utilized in conjunction with a container, such as a removable and/or disposable container, and, in certain embodiments, comprising of raised edges. The container may be filled with a waste receiving material, for example an absorbent, loose, granular material therein to capture excreta. The container must be of a certain size and dimension so that at least a portion fits within the housing.

The present disclosure details several embodiments of a litter box 1. In reference to FIG. 1, an embodiment details a litter box 1 that includes a housing 34 having a generally cuboidal configuration, although, non-limiting examples may include cube or prism shapes, or with one, two, or more open walls. In the embodiment of FIG. 1, the housing 34 has a top wall 23, a bottom wall 33, a front wall 22, a back wall 25, and two opposed side walls 24 and 32. The six walls connect at various edges, including the front left edge 2, the front top edge 3, the front bottom edge 4, the front right edge 5, the top left edge 6 the bottom left edge 7, the rear left edge 8, the rear top edge 9, the rear right edge 10, the top right edge 11, the bottom right edge 12, and the bottom rear edge 13. These vertices allow for connection of the walls, or for hingeable access as provided in other embodiments. The front wall 22 has an opening 15 therethrough to allow the entrance and exit of an animal into the interior of the housing 34. The housing 34 sits on legs 18, 19, 20, and 21 typically between 0.1 and 24, inches off the ground, more specifically between 1 and 5 inches, but the presence of the legs is not critical to the functioning of the litter box 1 and thus they may be excluded. Both the height of the legs and the size of the overall litter box 1 are of relevant consideration based on the species of animal to use a particular embodiment of the litter box, including but not limited to felines such as Felis lybica, Felis catus, and Felis nigripes of the genus Felis, dogs and wolves of the species Canis lupus, other small mammals such as weasels and ferrets, companion birds such as parrots, cockatoos, and doves, rodents such as mice, rats, hamsters, and guinea pigs, and reptiles such as lizards, snakes, and turtles as non-limiting examples.

The housing 34 is designed to accept and hold a removable and/or disposable container 16, which functions as a tray to hold a waste receiving material 17, such as many known loose granular materials (cat litter), wood shavings, paper, liners, absorbent beads or pulp, or other similar materials. The container 16 may rest on the top face 35 of bottom wall 33 of said housing 34, or otherwise be supported within the housing 34, and contain raised edges to maintain the waste receiving material 17 therein. The container 16 serves as a receptacle for holding waste receiving material 17 and wherein, an animal that enters the housing 34 to deposit its excreta, to be partially absorbed by the waste receiving material 17, inside the container 16. One embodiment of the container 16 is a disposable rectangular tray of a slightly smaller perimeter than that of the bottom wall 33 and height no taller than the bottom edge of the opening 15 in the housing 34. In certain embodiments, the top face 35 of said bottom wall 33 has at least one projection 70 of height equal to or less than the height of said container, said projections 70 affixed on each of the four corners of the top face 35 of said bottom wall 33 to retain said container 16 in place. Namely, there is a flange or retaining component on the four corners of the bottom wall 33, which aids in retaining the container 16 in place. Those with skill in the art will recognize that such containers can have variable size but due to some animals' habits of burying their excrement with a loose, granular material, the container 16 must be of sufficient depth to contain enough waste receiving material 17 to enable this habit.

One embodiment of the container 16 is composed of a biodegradable material that can be safely disposed of in a household garbage system and has a useful life of less than one month. This level of disposability allows for the convenience of the owner to dispose of the container 16 and waste receiving material 17 together once the waste receiving material 17 is too saturated with excreta for the animal to choose to enter the litter box any longer. One commercially available container which can be used is the LitterMaid Disposable Litter Box, model no-P-70000, made by LitterMaid, Edison, N.J. 08837, but a person having skill in the art will recognize containers of other materials, shapes, and sizes will perform the same function equally. One embodiment of the waste receiving material 17 is a clay-based, commercially available cat litter.

The composition of the waste receiving material 17 is not critical to the functionality of the litter box 1, notably with many variations suitable for use therein.

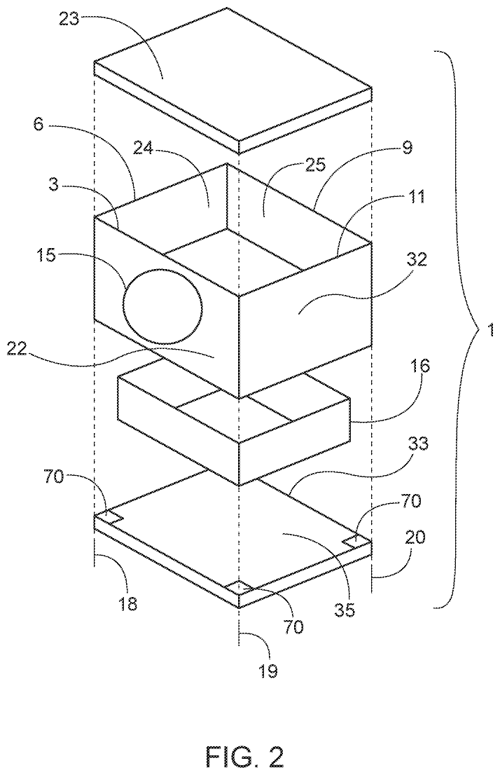

FIG. 2 details an exploded view of an embodiment of the litter box 1, detailing the components stacked upon one another. A top wall 23 is at the top, the front wall 22, side walls 24 and 32, and back wall 25 create a box, with an opening 15 in the front wall 22. The container 16 positioned to fit within the space created by the four walls 22, 24, 25, and 32, with edges 3, 6, 9, and 11 on the top, and the bottom of the walls, and all positioned on a bottom wall 33, with the top face 35 contacting the bottom face of the container 16.

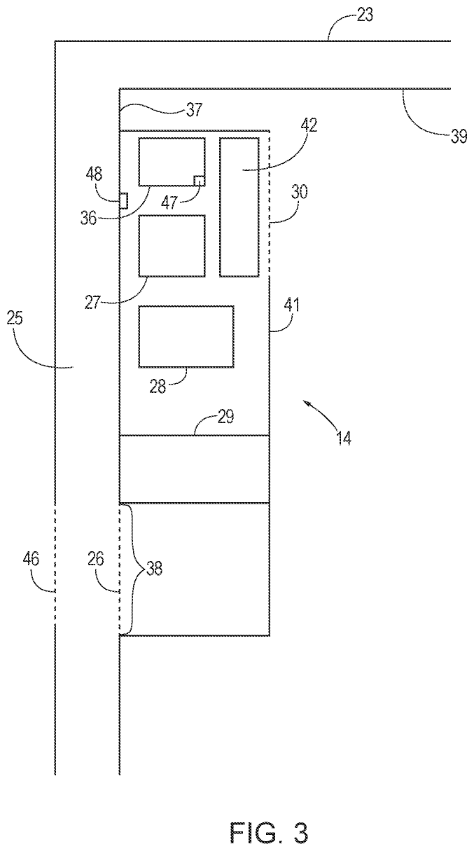

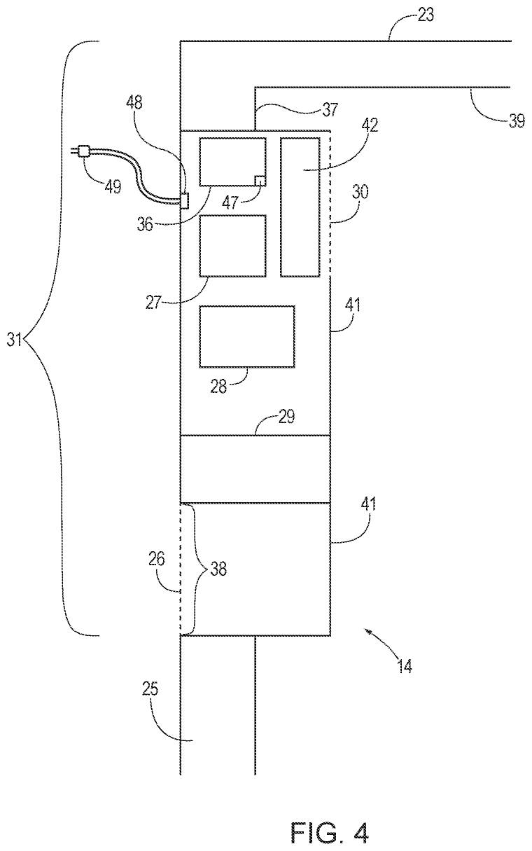

The removal of odors from the housing 34 is performed by an odor removal device 14. In an exemplary embodiment of the invention, the litter box 1 depicted in FIG. 3, the odor removal device 14 is affixed to the upper portion of the inside face 37 of the back wall 25 with one device exhaust vent 26 adjacent to a wall vent 46 therethrough the back wall 25 to allow for the escape of filtered air into the general air outside the housing 34. In an alternate embodiment depicted in FIG. 4, the odor removal device 14 is anchored in a wall opening 31 therethrough, e.g., in the back wall 25 and the air flows directly out the device exhaust vent 26 directly into general area outside the housing 34. Thus, the difference between FIGS. 3 and 4 is in FIG. 3, there is only a small wall vent 46, with the device placed on the inside face 37 of the wall, whereas in FIG. 4, there is a larger wall opening 31, which accepts the entire odor removal device 14. The device exhaust vent 26 may also be positioned to return air into the housing, so as to recirculate the air within the housing. The precise position of the odor removal device 14 may be modified based on the type of animal intended for use with the litter box 1, as well as ease of manufacture, and thus its precise placement can be easily modified without modifying the function of the invention. In certain embodiments, the odor removal device 14 is positioned at the top of the housing 34 to capture warmer odors (as the odors may be caused by warm excreta) to aid in removal of the odors.

A sensor 36 is utilized to activate the odor removal device 14. For example, the sensor is utilized to detect a change in the environment, such as it can be utilized to detect movement within the housing 34, namely, it detects the presence of an animal in the housing 34. The sensor 36 may be a motion sensor, heat sensor, image sensor, entry or exit sensor, light sensor, chemical sensor, pH sensor, weight sensor, a pressure sensor, volatile compound sensor, occupancy sensor, photo sensor, or other sensor capable of detecting the presence of an animal and activating the odor removal device 14. The sensor may sense one or two or more aspects from probes within the sensor, thus both light and pressure, for example may be sensed. Both the presence of an animal and the deposit of excreta may yield a number of environmental changes which can be detected by a sensor. In certain embodiments, a sensor may detect more than one parameter. One embodiment of the sensor 36 is an infrared motion sensor. One commercially available infrared motion sensor which can be used to make the sensor 36 is the Mini IR Pyroelectric Infrared Motion Sensor, model no-OP20, made by Shenzhen Setsail Co., Guangdong, Shenzhen, China. However, those with skill in the art will recognize that a range of sensors are suitable for the required function.

In some cases, the sensor 36 is operationally connected with a timer 47 to activate the fan 27 and/or the plasma generating unit 28 on a timed delay after the sensor 36 has detected an animal exiting the housing 34. By operationally connected, this means that the connection is made via circuitry, wireless connectivity, and/or programable instructions for operating electrical elements. Therefore, the sensor 36 acts in conjunction with a timer 47 or timing program/instructions to activate at least one or both of, the fan 27 and plasma generating unit 28. One embodiment has the fan 27 and the plasma generating unit 28 activate a few seconds, such as 30 seconds or between 1 and 60 seconds, after it has detected that an animal has entered and exited the housing 34. Alternate embodiments activate the fan 27 and the plasma generating unit 28 between one and three minutes after the exit of the animal. The sensor 36 can be used to also detect the first movement or presence within the housing 34, and then only activates upon the absence of movement or removal of the animal's presence from within the housing 34, i.e., the animal has exited the litter box 1, and no movement is now present inside of the litter box 1. In other embodiments, a setting of High/Medium/Low can be provided to the owner of the litter box 1 to adjust the time of the odor removal device's 14 operation to better suit their animal's behavior and the amount of odor the animal generates. In such instances a high odor would initiate operation within a shorter amount of time than a medium or low setting and/or operate for a longer duration. The sensor 36 may, therefore, also be an odor sensor, a pH sensor, or volatile compound sensor, an image sensor, or others which may help to identify the excreta and modify the setting of the device. Similarly, the device may operate for as long as the sensor 36 is detecting an odor within the unit.

The fan 27 and the plasma generating unit 28 must be activated long enough after the animal has exited the housing 34 so as not to deter the animal from utilizing the litter box 1 by the sound of the odor removal device 14, while not waiting too long after the exit of the animal that the odors produced by the animal can escape through the opening 15 into the general area outside of the litter box 1. In this embodiment, fan 27 and the plasma generating unit 28 would then run for between thirty to sixty seconds before shutting off to eliminate the odor producing chemicals released into the air of the housing 34 by an animal after it deposits its excreta in the container 16. Alternate embodiments of the sensor 36 contain a timer 47 capable of activating the fan 27 and the plasma generating unit 28 to run for an interval of between one and five minutes, or longer, as needed, before shutting off. Another embodiment of the sensor 36 timer allows for preset time delay intervals for the fan 27 and the plasma generating unit 28 to activate to eliminate newly produced odor producing chemicals within the housing 34 produced as excrement that has not yet been removed from the housing 34 decomposes, or as detected by the sensor 36. The fan 27 may be part of the odor removal device 14, or separately disposed of with the housing 34, to function with the odor removal device.

The odor removal device 14, used to remove odors produced by an animal that uses the litter box 1, contains a sensor 36, a fan 27, a plasma generating unit 28, a pre-filter 42, and a silver-impregnated, activated carbon filter 29 housed within a box 41. The box 41 contains an inlet air vent 30 built in one portion of one wall facing the interior of the housing 34 of the litter box 1 for to allow for the entrance of air into the odor removal device 14. The box 41 contains an exhaust vent 38 built into the wall of the box 41 to allow for air to exit the box. The exhaust vent 38 in one embodiment, depicted in FIG. 3 is adjacent the odor removal device vent 26 built therethrough one wall of the housing 34 to allow for the escape of air to the general air outside the box 41, or litter box through the wall vent 46. In another embodiment, depicted in FIG. 4, the odor removal device 14 is affixed into a wall opening 31 the size of the odor removal device 14 cut therethrough one wall of the housing 34. In this embodiment, the air that travels through the odor removal device 14 exits out the exhaust vent 38 directly into the general area outside the housing 34. The precise positioning of the air inlet vent 30 and exhaust vent 38 is dependent on the position of the odor removal device 14. The entirety of the odor removal device 14 may be removable from the housing 34 as to allow it to be cleaned, parts exchanged, or to be inserted or replaced into a different housing. Thus, a housing and the odor removal device can be separately manufactured. In certain embodiments, the housing may be a room, for example a toilet room.

In certain embodiments, the odor removal device 14 is operated for a pre-determined amount of time to remove an odor. However, the odor removal device 14 thereafter periodically operates by reactivating the fan 27 and/or the plasma generating unit 28 for a subsequent pre-determined time interval. This can be until the sensor 36 detects a new change in the environment, i.e., entry of an animal, or some other environmental change. This allows for an initial cleaning of the air/odor, but periodic reactivation of the odor removal device to ensure that any lingering or newly created odors are also addressed.

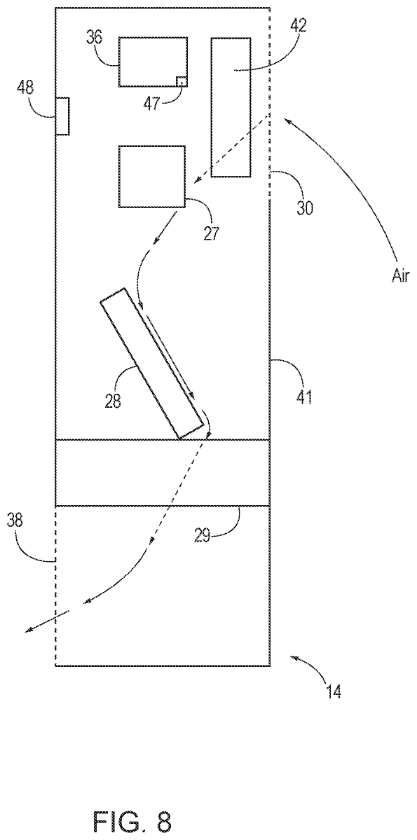

As depicted in the exemplary diagram in FIG. 8, a fan 27 moves air depicted by the arrows in the figure containing odor producing chemicals into the odor removal device 14, housed in box 41, from the inside of the housing 34. The fan 27 is activated and turns on in response to a signal from the sensor 36 that detects the entrance and exit of an animal. An embodiment of the fan 27 is a motorized fan with an air flow range of 10-100 cubic feet per minute (CFM), a CFM range which is adequate for most household uses. One commercially available fan suitable for this purpose is the aluminum, 1.2 W, 18 dba, 30 CFM, computer fan model no. TWFM0090 made by Twister Commercial Co. of Guangdong, Shenzhen, China. The size and airflow capability of the fan 27 may vary with the size of the animal to use a particular embodiment of the litter box 1.

The fan 27 pulls air in through the air inlet vent 30 in the odor removal device 14 box 41 and through an optional pre-filter 42 placed adjacent to the air inlet vent 30. The pre-filter 42 is a made of a porous material that is permeable enough to allow for the flow of air containing odor producing chemicals through the pre-filter but impermeable enough to stop the travel of larger particles traveling through the air such as hair, fur, feathers, dander, and large dust particles. The pre-filter 42 protects the interior components of the odor removal device 14, particularly mechanical components such as the fan 27 and the plasma generating unit 28, from collection of and interference from larger particles in the air that could otherwise travel in the air through the odor removal device 14. The pre-filter 42 will need to be cleaned manually by the user regularly to allow for the continued flow of air through the odor removal device 14 and will need to be completely replaced periodically during the life of the odor removal device 14. The user will be instructed on the appropriate cleaning and replacement cycles.

Simultaneous with the activation of the fan 27, the plasma generating unit 28 also turns on in response to the signal from the sensor 36. The plasma generating unit 28 then produces a certain concentration of ozone for the duration of the activation of the odor removal device 14. The plasma contains reactive species including but not limited to: ozone (O₃), hydrogen peroxide (H₂O₂), electronically excited oxygen (O₂*), hydroxyl radical (OH), nitric oxide (NO), peroxynitrite radical (ONOO*), and other reactive oxygen and reactive nitrogen species. These reactive species break down and eliminate the odor producing chemicals present in animal excreta, including volatile organic compounds, ammonia, and hydrogen sulfide (H₂S), which are carried in the air passing through the odor removal device 14. The reaction produces byproducts of carbon dioxide (CO₂), water (H₂O), and trace amounts of nitrogen oxides (NOₓ), sulfur oxides (SOₓ), hydrogen peroxide (H₂O₂), and ozone (O₃). In an embodiment, the plasma generating unit 28 contains a ceramic plate that allows for the impinging of air onto the plasma surface discharge, allowing for the improved removal of volatile organic compounds. One commercially available plasma generating unit is a 200 mg/h, 5W, 3.0-3.5 KV-18 KHz ozone generator, model no- KHP-200MGLA1, made by DGOzone, Ltd. of Shanghai, China which can be used to make the plasma generating unit 28. Those having skill in the art will realize that other commercially available ozone generators are available for this function and that the power and size of the plasma generating unit 28 will vary with the size of the animal using the particular embodiment of the invention.

To eliminate the byproducts produced by the plasma generating unit 28, including NOₓ, SOₓ, and O₃, that that can induce adverse health effects in living species near the device, a silver-impregnated activated carbon filter 29 is built into the odor removal device 14 below the plasma generating unit 28. As depicted in the exemplary diagram in FIG. 8, the air that has been pulled into the box 41 of the odor removal device 14 by the fan 27 enters through the air inlet vent 30, flows through the pre-filter 42, down around the plasma generating unit 28, then flows through the activated carbon filter 29 and finally out the box 41 through exhaust vent 38. Activated carbon dissociates O₃ and H₂O₂ into oxygen and water, traps NOₓ and SOₓ on its surface, and also traps any bacteria, fungi, and viruses that might be present in the air flow through the odor removal device 14. In certain embodiments, the activated carbon is a silver-impregnated activated carbon and wherein the silver ions present in the silver-impregnated activated carbon filter 29 also help eliminate bacteria, keeping the activated carbon filter 29 usable for longer periods of time. The activated carbon filter 29, however, will eventually run out of active sites for absorption of new materials and will need to be replaced periodically. A commercially available filter that can be used for the activated carbon filter 29 is a Viro-Silver Replacement Pre-Filter Sheet, model no-F1700 made by Hunter Pure Air, Fort Wayne, Ind. Those having skill in the art will recognize that other commercially available silver-impregnated, activated carbon filters, or another activated carbon material, or other material suitable to capture byproducts from the ozone generator and excreta, as known to those of ordinary skill in the art and can perform the same function, regardless of the manner in which such materials are captured.

The components of the odor removal device 14, including the sensor 36, the fan 27, and the plasma generating unit 28 must have an electrical connection to operate. Thus, the device is connected to a power source 48, whether a DC source or an AC source. An embodiment of the litter box 1 utilizes a power alternating current (AC) power plug 49 to power the odor removal device 14. Alternate embodiments utilize disposable alkaline batteries or nickel-metal hydride (NiMH) rechargeable batteries as the power source 48 the odor removal device 14 or other energy sources to provide the AC or DC power.

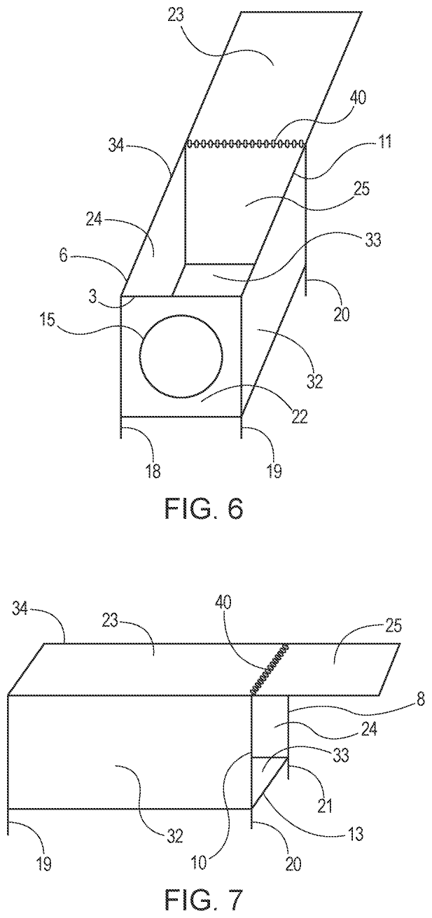

In an embodiment of the present invention a litter box 1, depicted in FIG. 6, the inside of the housing 34 is accessible by the pulling away of the top wall 23 outward from the housing 34. This is achieved by the top wall 23 being attached to back wall 25 by a hinge mechanism 40 along linear side 9 and detached from the other three adjacent walls, 24, 22, and 32. Therefore, the top wall 23 is disconnected from each of the top edges 3, 6, and 11, on the adjacent walls 24, 22, and 32. This opening of the top wall 23 allows for removal and replacement of the container 16. In an alternate embodiment, depicted in FIG. 7, the back wall 25 is attached to top wall 23 by a hinge mechanism 40 along linear edge 9 and detached from adjacent walls 24, 33, and 32, allowing for the opening of the back wall 25 away and outward from the housing 34. In this manner, the back wall 25 opens and closes the device, and no opening 15 is required in the front wall 22, which may assist in containing odor. This would require the movable wall to operate in the presence of an animal (such as via a sensor) to allow for ingress and egress from the device.

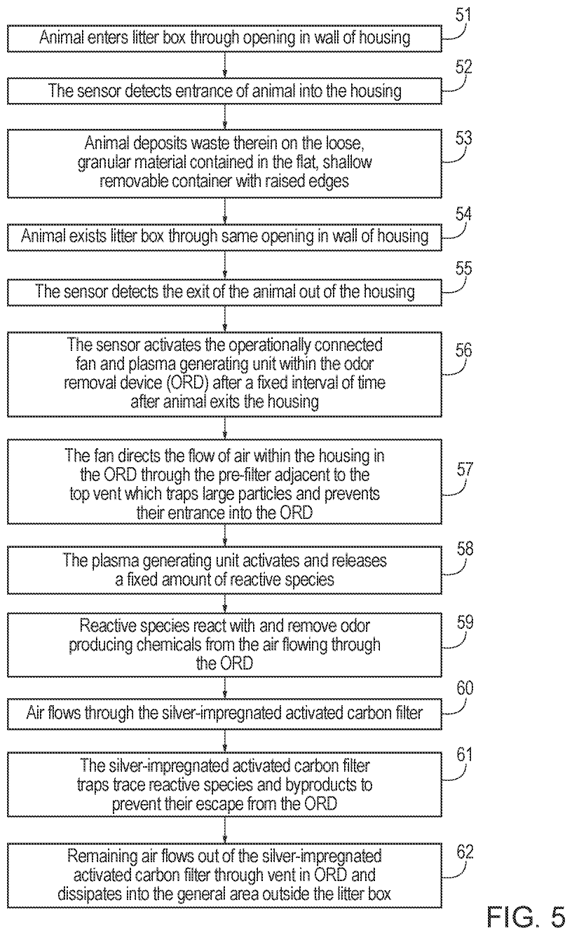

FIG. 5 details one embodiment of use of the device for processing odors generated from animal waste. The animal enters the litter box 51, the sensor detects the animal's entrance 52. The animal deposits waste within the container 52, and the animal exits 54. The sensor detects the exit or lack of presence of the animal 55, and the sensor operationally activates the fan and/or the plasma generating unit upon a fixed interval of time 56. The fan pushes or pulls air through a pre-filter to capture large particles 57 and the activated plasma unit, generates reactive species 58 which react with and neutralize odor producing chemicals 59. Air flows through a carbon filter 60 and, in this embodiment, the silver impregnated filter traps trace reactive species to prevent their escape 61. Finally, the air flows out of the exhaust vent to dissipate into the areas outside of the litter box 62.

In certain embodiments, the sensor 36 is able to differentiate between two different animals. In the simplest example, the sensor 36 would use an image or pressure recognition to differentiate a child from a parent by image or pressure, for example. The sensor 36 will then provide a signal or instructions to the odor removal device 14 to operate in a different manner based on the difference signal received from the sensor 36. For example, a larger animal may create greater amounts of odors and so the fan may operate at a faster rate, it may operate for a longer period, the plasma generator may run for a longer time or at a higher power as compared to a smaller animal. Thus, the devices herein are able to differentiate between one signal from another and to operate the odor removal device 14 differently based upon that different signal received from the sensor 36.

Although the devices and methods have been described with respect to specific embodiments, it is not intended to be limited thereto and various modifications which will become apparent to the person of ordinary skill in the art are intended to fail within the spirit and scope of the invention as described herein taken in conjunction with the accompanying drawings and the appended claims.

What is claimed is:

1. An animal litter box, comprising:
a housing having an opening allowing for entry d exit of an animal;
a sensor to detect a change in an environment of an interior of the housing, wherein the sensor is a motion sensor, a moisture sensor, an image sensor, a heat sensor, a pH sensor, a light sensor, a weight sensor, a pressure sensor, entry or exit sensor, occupancy sensor, or a photo sensor; and
an odor removal device operationally connected to the sensor such that the odor removal device is activated in response to a signal from the sensor, and wherein the odor removal device is configured to operate in different manners based on respectively different signals from the sensor.

2. The animal litter box of claim 1, wherein the odor removal device comprises a plasma generating unit and a motorized fan for passing air into the odor removal device.

3. The animal litter box of claim 2, wherein the motorized fan is configured to operate at different speeds and/or for different durations based on respectively different signals from the sensor.

4. The animal litter box of claim 2, wherein the plasma generating unit configured to operate with different power levels and/or for different durations based on respectively different signals from the sensor.

5. The animal litter box of claim 1, wherein the odor removal device comprises an ozone generator.

6. The animal litter box of claim 1, wherein the sensor is configured to provide the respectively different signals based on differentiating characteristics of different animals detected by the sensor.

7. The animal litter box of claim 1 wherein the sensor is configured to provide the respectively different signals based on different types of excreta detected by the sensor.

8. An animal litter box comprising:
a housing having an opening allowing for entry and exit of an animal;
a sensor to detect a change in an environment of an interior of the housing, -herein the sensor is a moisture sensor or a pH sensor; and
an odor removal device operationally connected to the sensor such that at least one component of the odor removal device is activated in response to a signal from the sensor.

9. The animal litter box of claim 8, wherein the odor removal device comprises a plasma generating unit and a motorized fan for passing air into the odor removal device.

10. The animal litter box of claim 9, wherein the at least one component of the odor removal device is the motorized fan.

11. The animal litter box of claim 9, wherein the at least one component of the odor removal device is the plasma generating unit.

12. The animal litter box of claim 8, wherein the odor removal device comprises an ozone generator.

13. An animal litter box, comprising:
a housing having an opening allowing for entry and exit of an animal;
a sensor to detect a change in an environment of an interior of the housing;
an odor removal device; and
tuner configured with the sensor for activating and periodically reactivating the odor removal device at preset time intervals in response to a signal from the sensor.

14. The animal litter box of claim 13, wherein the timer is configured to periodically reactivate the odor removal device at preset time intervals until the sensor detects a new change in the environment

15. The animal litter box of claim 13, wherein the odor removal device comprises a plasma generating unit.

16. The animal litter box of claim 13, wherein the odor removal device comprises an ozone generator,

17. An animal litterbox, comprising:
a housing having an opening allowing for entry and exit of an animal;
a sensor to detect a change in an environment of an interior of the housing;
an odor removal device operationally connected to the sensor; and
a timer having user adjustable settings of different durations for which e odor removal device is operated in response to a signal from the sensor.

18. The animal litter box of claim 17, wherein the user adjustable settings further include different time periods at which operation of the odor removal device is initiated in response to a signal from the sensor.

19. The animal litter box of claim 17, wherein the odor removal device comprises a plasma generating unit.

20. The animal litter box of claim 17, wherein the odor removal device comprises an ozone generator.

21. An animal litter box, comprising:
a housing having an opening allowing for entry and exit of an animal;
a sensor to detect a change in an environment of an interior of the housing;
an odor removal device operationally connected to the sensor; and
a timer having user adjustable settings of different time periods at which to initiate activation of the odor removal device in response to a signal from the sensor.

22. The animal litter box of claim 21, wherein the odor removal device comprises a plasma generating unit.

23. The animal litter box of claim 21, wherein the odor removal device comprises an ozone generator.
